(19) United States
(12) Patent Application Publication
(10) Pub. No.: US 2007/0148192 A1
(43) Pub. Date: Jun. 28, 2007

(54) Stable ophthalmic composition

(76) Inventors: Ritu Nitin Laddha, Akota (IN); Subas Balaram Bhowmick, Akota (IN)

Correspondence Address: MERCHANT & GOULD PC, P.O. BOX 2903, MINNEAPOLIS, MN, 55402-0903, US

(21) Appl. No.: 10/546,417

(22) PCT Filed: Feb. 23, 2004
(86) PCT No.: PCT/IN04/00048

§ 371 Date: May 22, 2006

(30) Foreign Application Priority Data
Feb. 21, 2003 (IN) 212/MUM/2003

(51) Int. Cl. A61K 31/724; A61K 31/7034; A61K 31/573; A61K 31/496

(52) U.S. Cl. 424/400; 514/58; 514/35; 514/171; 514/253.08

(57) ABSTRACT

The present invention provides a clear stable ophthalmic composition comprising (a) an anti-infective agent; (b) a steroidal anti-inflammatory agent; (c) a complexing agent capable of forming an inclusion complex and (d) other pharmaceutically acceptable excipients in a liquid vehicle such that the composition is free of any other complexation enhancing polymer and such composition when stored at room temperature for one year does not show any precipitation over the storage period.

The present invention relates to a stable ophthalmic composition comprising an anti-infective agent and an anti-inflammatory agent.

### BACKGROUND OF THE INVENTION

Formulations containing an anti-infective agent and an anti-inflammatory agent such as corticosteroid are useful for topical application to the eye, ear, nose or skin. Corticosteroids are insoluble in water and thus are generally available in suspended form or are dissolved in oil or solvents when used in the formulation. However, for ophthalmic use it would be desirable to avoid the use of oil or solvents and provide a clear solution of the anti-infective and the corticosteroid in a predominantly aqueous phase and a formulation in suspension form causes discomfort to the eye and requires inclusion of many additional excipients to formulate a stable suspension composition which may also cause irritation to the eye. Thus a clear solution composition is the most preferred formulation for administration to the eye.

Most of the combination therapy of anti-infective and anti-inflammatory agents for ophthalmic treatment is available commercially in a suspension form. For example tobramycin, an aminoglycoside derivative and dexamethasone, a corticosteroid is available as an antibiotic and steroid combination for topical ophthalmic use in a suspension form, ciprofloxacin, a broad-spectrum antibacterial is commercially available in combination with a cortisosteroid like dexamethasone in India for the treatment of ocular infections and inflammations in a suspension form. Dexamethasone has a very low solubility in water and is thus generally formulated in a suspended form. It can also be prepared as solutions at relatively higher pH values, however if a solution of ciprofloxacin HCl is mixed with this solution, in case of formulating a combination therapy for ocular treatment, precipitation of ciprofloxacin occurs and the solution becomes turbid. A stable ophthalmic solution composition may be expected to have better patient compliance and acceptance as compared to the available suspension formulation. Hence it is the object of the present invention to provide a clear, stable ophthalmic solution composition of a combination of an anti-infective agent and an anti-inflammatory steroid.

U.S. Pat. No. 4,844,902 assigned to Bayer, Germany, claims a topically applicable formulation comprising by weight about 0.01 to 30% of an anti-bacterially active compound, 0.01 to 10% of a corticosteroid and a carrier. The invention relates to topically applicable formulations for the treatment or prophylaxis of infections, diseases and injuries to the skin, including bums. The topical formulations include solutions, sprays, lotions, gels, ointments, creams, powders, dusting powder sprays, pastes, suspensions, emulsions, foams and sticks containing the active compounds. The solution compositions exemplified in this patent are not ophthalmic compositions because they use solvents that may be unacceptable for ophthalmic use.

PCT application WO 90/01933 assigned to Alcon laboratories claims an antibiotic/antiinflammatory ophthalmic pharmaceutical composition for topical delivery to the eye. This patent discloses pharmaceutical compositions containing a quinolone antibiotic, such as ciprofloxacin and a steroid such as rimexolone, dexamethasone, fluorometholone and the like for topical ophthalmic delivery. The exemplified compositions are suspension composition and ointment for ophthalmic use, thus this patent does not disclose a clear, stable solution composition of an antibiotic and corticosteroid as provided in the present invention.

U.S. Pat. No. 6,359,016 assigned to Alcon Universal Ltd., relates to topical suspension formulations containing ciprofloxacin and dexamethasone. Specifically the invention relates to stable suspension formulations of ciprofloxacin and dexamethasone that lack a nonionic tonicity agent, such as glycerol or mannitol. Thus this patent does not provide means of developing a clear, stable solution composition for a combination of an anti-bacterial and an anti-inflammatory agent, which is the most preferred composition for administration to the eye. United States Patent Application No. 20010049366 assigned to Alcon Universal Ltd, relates to topical solution formulations containing an antibiotic, a corticosteroid and a solubilizing agent and they claim a topically administrable solution composition intended for application to the eye, ear, nose or skin comprising a) 0.01-1% (w/v) of a corticosteroid; b) 0.1-1.5% (w/v) of an antibiotic drug; c) a vitamin E tocopheryl derivative in an amount sufficient to solubilize the corticosteroid; d) a tonicity agent in an amount sufficient to cause the composition to have an osmolality of about 250-350 mOsm; and e) a buffering agent. Thus the solubilizing agent in the formulation of the patented invention is a vitamin E tocopherol and the patent does not provide formulations wherein cyclodextrin is used to solubilize the corticosteroid.

PCT application WO 02/39993 claims a stable pharmaceutical preparation of a combination drug, comprising amongst others of (i) an anti-infective agent, selected from the group consisting of quinolone derivatives, amino-glycoside derivatives and their pharmaceutically acceptable salts; (ii) an ant-inflammatory agent which is a corticosteroid; (iii) a complexation enhancing polymer; (iv) a solubilizer exhibiting an inclusion phenomena; (v) pharmaceutically acceptable excipients within a suitable carrier system. The invention exemplifies an eye drop solution formulation of dexamethasone and ciprofloxacin, wherein beta-cyclodextrin is added as the solubilizer exhibiting an inclusion phenomena and polyvinyl alcohol is added as the complexation-enhancing polymer. The patent thus teaches the preparation of solutions of the anti-infective agent and the anti-inflammatory agent with the use of an inclusion complex forming solubilizer in presence of a complexation-enhancing polymer. Use of a complexation-enhancing polymer, however, effectively reduces the amount of uncomplexed drug in solution. A higher amount of drug available in the free uncomplexed form would be desirable.

### Object of the Invention

Thus the objects of the invention are—
1) To provide ophthalmic composition of an anti-infective agent and an anti-inflammatory agent.
2) To provide an ophthalmic composition comprising an anti-infective agent and an anti-inflammatory agent in a liquid vehicle along with other pharmaceutically acceptable excipients.
3) To provide the aforesaid solution composition of an anti-infective and an anti-inflammatory agent, which is clear and stable.
4) To provide the aforesaid solution composition which is stabilized against chemical degradation and there is no precipitation observed during the storage period.
5) To provide aforesaid solution composition wherein the corticosteroid is solubilized by addition of complexing agent capable of forming an inclusion complex.
6) To particularly provide the aforesaid solution composition wherein cyclodextrin is used as the complexing agent capable of forming an inclusion complex without the inclusion of any other complexation enhancing polymer.
7) To provide a clear, stable aqueous solution composition of an anti-infective and a steroidal anti-inflammatory which when administered topically to the eye does not cause any irritation/discomfort to the eye.
8) To provide aforesaid ophthalmic solution composition having more patient compliance and acceptability.
9) To provide aqueous solution composition of a combination of an anti-infective and an anti-inflammatory agent having a high availability of the actives at the site of action.
10) To provide ophthalmic solution composition of an anti-infective agent like ciprofloxacin hydrochloride in combination with a steroidal anti-inflammatory agent like dexamethasone.
11) To provide clear stable aqueous solution composition of ciprofloxacin and dexamethasone comprising a complexing agent like β cyclodextrin and other pharmaceutically acceptable excipients having a pH not exceeding 6.5.

### SUMMARY OF THE INVENTION

Thus the present invention provides a clear, stable ophthalmic composition comprising (a) an anti-infective agent; (b) an anti-inflammatory agent and (c) a complexing agent capable of forming an inclusion complex.

The present invention particularly provides a clear, stable ophthalmic composition comprising (a) an anti-infective agent; (b) a steroidal anti-inflammatory agent; (c) a complexing agent capable of forming an inclusion complex and (d) other pharmaceutically acceptable excipients in a liquid vehicle such that the composition is free of any other complexation enhancing polymer and such composition when stored at room temperature for one year does not show any precipitation over the storage period.

The said anti-infective agent of the present invention is a quinoline derivative like ciprofloxacin hydrochloride or an aminoglycolide derivative like tobramycin and the anti-inflammatory agent is a corticosteroid like dexamethasone.

### DETAILED DESCRIPTION OF THE INVENTION

According to the invention we provide a topically administrable solution formulation containing an anti-infective agent, a steroidal anti-inflammatory agent, a complexing agent capable of forming an inclusion complex and other pharmaceutically acceptable excipients.

The said actives and excipients are incorporated in a liquid vehicle consisting of water resulting in a clear stable solution for ocular treatment of infection and inflammation.

The anti-infective agent may be any ophthalmically useful quinolone derivative or an aminoglycoside derivative. The quinolone derivative that may preferably be used in the present invention include fluoroquinolones from the group consisting of ciprofloxacin, moxifloxacin, gatifloxacin, gemifloxacin, norfloxacin, ofloxacin, levofloxacin and trovofloxacin, its pharmaceutically acceptable salts and the like and the aminoglycoside derivative may be selected from the group consisting of tobramycin, gentamycin and its pharmaceutically acceptable salts. The preferred quinolone anti-infective ingredient of the present invention is ciprofloxacin, a fluoroquinolone anti-infective, active against a broad spectrum of gram-positive and gram-negative ocular pathogens and the preferred form is ciprofloxacin hydrochloride. It is available as the monohydrochloride monohydrate salt of 1-cyclopropyl-6-fluro-1,4-dihydro-4-oxo-7-(1-piperazinyl)-3-quinoline-carboxylic acid. The preferred aminoglycoside antibacterial anti-infective agent of the present invention is tobramycin, chemically which is O-3-deoxy-α-D -glucopyranosyl-( 1-6)-O-[2,6-diamino-2,3,6-trideoxy-α-D-ribo-hexopyranosyl-( 1-4)] -2-deoxy-D-streptamine.

The anti-inflammatory agents that may preferably be used in the present invention selected from a corticosteroid include flurometholone, betamethasone, prednisolone, dexamethasone, their derivatives and the like. The preferred corticosteroid ingredient of the present invention is dexamethasone and/or its derivatives. Dexamethasone is a synthetic analog of naturally occurring glucocorticoids (hydrocortisone and cortisone). It is available as dexamethasone alcohol, dexamethasone acetate, dexamethasone sodium phosphate and the like.

The anti-infective agent included in the stable ophthalmic composition of the present invention will comprise about 0.1 to 30% weight/volume units and the steroidal anti-inflammatory agent will comprise about 0.01 to 10% weight/volume units. In preferred embodiments wherein the anti-infective is ciprofloxacin HCl and the corticosteroid is dexamethasone the amount ranges from 0.1 to 1.5% w/v and 0.01 to 1.0% w/v respectively.

In addition to an anti-infective agent and a corticosteroid the stable ophthalmic composition of the present invention comprises a complexing agent capable of solubilizing the steroid in water by forming an inclusion complex. The complexing agents selected in the present invention may be a cyclodextrin and its derivatives which can be well tolerated when administered by ophthalmic route for e.g. α-, β- or γ-cyclodextrins or derivatives thereof, preferably derivatives wherein one or more of the hydroxy groups are substituted, e.g. by alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl groups, wherein each alkyl or alkylene moiety preferably contains up to six carbons. Substituted cyclodextrins, which can be used in the present invention, include ethers, polyethers or mixed ethers thereof. More preferably such substituted cyclodextrins are ethers wherein the hydrogen of one or more cyclodextrin hydroxy groups is replaced by one or more cyclodextrin hydroxy groups is replaced by C₁₋₃alkyl, hydroxy-C₂₋₄alkyl or carboxy-C₁₋₂alkyl or more particularly by methyl, ethyl, hydroxyethyl, hydroxypropyl, hydroxybutyl, carboxymethyl or carboxyethyl. Most preferred cyclodextrins used in the present invention are β-cyclodextrin ethers and polyethers e.g. dimethyl-β-cyclodextrin, hydroxypropyl-β-cyclodextrin and hydroxyethyl-β-cyclodextrin. In the stable ophthalmic composition of the present invention, the cyclodextrin is preferably used at 0.05% w/v to about 15.0% w/v, more preferably at 1.0% w/v to about 10.0% w/v and most preferably about 1.5% w/v to about 5.5% w/v.

In preferred embodiments of the stable ophthalmic composition of present invention hydroxypropyl-β-cyclodextrin is used. Commercially, Hydroxypropyl-β-cyclodextrin is made by reacting β-cyclodextrin with propylene oxide, it has a true density of 1.378 g/cm³ and has a cavity diameter of 6.0-6.5 Å.

In the present invention cyclodextrin is present in an amount sufficient to solubilize the corticosteroid ingredient. In addition to this in prior art compositions complexation enhancing agents consisting of non-ionic polymers has been used in the preparation of cyclodextrin-drug complexes as a means for increasing the solubilizing and stabilizing effects of cyclodextrin derivatives on drugs and complexation therewith. In the present invention a clear and stable solution composition is provided without the inclusion of such additional polymers by a simple process of incorporating the actives and other excipients in a liquid vehicle to obtain a composition which when stored at room temperature for one year does not show any precipitation over the storage period and the active agents are also stabilized against their chemical decomposition.

Further the stable ophthalmic composition of the present invention may contain other ophthalmically acceptable excipients, e.g. osmogens, chelating agents, preservative agents, buffers etc. present in an amount ranging from 0.01% w/v to about 99.9% w/v.

Examples of the osmogent(s) that may be used in the stable ophthalmic composition of the present invention include inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; sodium carbonate or sodium bicarbonate; carbohydrates such as mannitol, sorbitol, arabinose, ribose, xylose, glucose, dextrose, fructose, mannose, galactose, sucrose, maltose, lactose, raffinose, inositol, xylitol, maltitol; water-soluble amino acids such as glycine, leucine, alanine, or methionine; urea and the like, and mixtures thereof. These basically include all pharmaceutically acceptable and pharmacologically inert water-soluble compounds referred to in the pharmacopoeias such as United States Pharmacopoeia, as well as in Remington: The Science and Practice of Pharmacy; edition 19; Mack Publishing Company, Easton, Pa. (1995). Pharmaceutically acceptable water-soluble salts of inorganic or organic acids, or non-ionic organic compounds with high water solubility, e.g. carbohydrates such as sugar, or amino acids, are generally preferred. The osmogent may be added in an amount, which renders the solution isoosmotic. Mannitol is used in the present invention as the preferred osmogent.

If desired, chelating agents may be used in the stable ophthalmic composition of the present invention and are selected from a group comprising edetic acid, edetic acid salts like disodium edetate, sodium edetate, edetate calcium disodium and trisodium edetate, malic acid and the like and mixtures thereof. Chelating agents if present is used in an amount from about 0.001 to 0.1% w/v. In preferred embodiments disodium edetate is added at concentration of 0.05% w/v.

The stable ophthalmic compositions of the present invention may be prepared without a preservative as a “unit-dose” or “unpreserved” formulation or as “preserved” or “multi-dose” formulation. The multi-dose compositions may contain an ophthalmically acceptable preservative.

The preservative agent that may be added in the stable ophthalmic composition of the present invention to protect the solution composition from microbial contamination are selected from the group comprising benzalkonium chloride, methyl paraben, propyl paraben and their salts, potassium sorbate and sodium benzoate. The preservative may be present in an amount ranging from about 0.002 to 0.5% w/w of the formulation. The preferred preservative used in the present invention is benzalkonium chloride.

The stable ophthalmic composition of the present invention may additionally contain ophthalmically acceptable solubilizing agent that solubilize the drug while maintaining its availability in a free uncomplexed form. The solubilizer is such that it does not include the drug in an inclusion cavity. Examples of such a solubilizer include cosolvents, complexing agents that form a rapidly dissociating complex, surfactants, and hydrotropic agents.

The pH of the stable ophthalmic composition of the present invention may be adjusted using suitable pH adjusting agents, selected from a group of buffering agents, comprising lactic acid, citric acid, tartaric acid, phosphoric acid, acetic acid, hydrochloric acid, nitric acid, sodium or potassium metaphosphate, sodium or potassium phosphate, sodium or potassium acetate, ammonia, sodium carbonate, sodium or potassium hydroxide, dibasic sodium phosphate, sodium borate, and the like and mixtures thereof. Adjusting pH using strong mineral acids like hydrochloric acid is more preferred to a pH less than 6.5.

In preferred embodiment where ciprofloxacin HCl is the anti-infective agent and dexamethasone the anti-inflammatory agent the stable ophthalmic composition is adjusted to a pH preferably in the range between about 3.5 and 6.0, more preferably between about 4.0 and 5.0 and most preferably between about 4.4 and 4.6.

The clear, stable solution composition may be administered topically to the eye as well as if desired may be administered to the ear, nose or skin.

The stable ophthalmic composition of the present invention may be prepared by mixing the two drugs and other pharmaceutically acceptable excipients in purified water. The solution pH may be adjusted if necessary. In specific embodiments comprising dexamethasone and ciprofloxacin HCl the pH is about 4.5. The solutions thus obtained may be filled in suitable containers such as multidose vials with addition of preservatives or as unit dose vials without a preservative. The ophthalmic compositions may be sterilized by any techniques used in art, preferably by filtration.

In a preferred embodiment, the stable ophthalmic composition of the present invention may be prepared by a simple process comprising dissolving mannitol in water for injection, adding hydroxypropyl β-cyclodextrin in small increments to the above solution and stirring to get a clear solution. Dexamethasone is then added to this solution and stirred to get a clear solution. Ciprofloxacin first and then disodium edetate and benzalkonium chloride solution is added and dissolved to get a clear solution and volume made up with water for injection. The solution may be filtered to obtain a clear stable ophthalmic composition, which is then filled in vials.

The invention is illustrated by the following examples, which is by no means intended to limit the scope of the invention but is given by way of illustration.

The stable ophthalmic composition of the present invention can be made as shown in the example given below.

Example 1

TABLE 1

| Sr. No. | Ingredients | mg/mL | Percent (w/v) |
|---|---|---|---|
| 1 | Ciprofloxacin HCl eq. to | 3.0 | 0.3 |
| | Ciprofloxacin | | |
| 2 | Dexamethasone | 1.0 | 0.1 |
| 3 | Mannitol | 40.0 | 4.0 |
| 4 | Hydroxypropyl β-cyclodextrin | 27.01 | 2.701 |
| 5 | Disodium edetate | 0.5 | 0.05 |
| 6 | Benzalkonium chloride solution | 0.1 | 0.01 |
| 7 | Water for Injection quantity | 1.0 mL | 100 |
| | sufficient to make | | |

Mannitol is dissolved in water for injection, hydroxypropyl β-cyclodextrin is added in small increments to this solution and stirred to get a clear solution. Dexamethasone is added to this solution and stirred to get a clear solution. Ciprofloxacin is then added to the solution and stirred till it is clear. Disodium edetate and benzalkonium chloride solution is added and dissolved to get a clear solution. The pH of the solution was 4.5. After making up the volume with water for injection, the solution is filtered and filled in vials.

The solutions were subjected to stability studies by storing in sealed vials at 25° C., 40° C. and 50° C. storage conditions. The initial, 3 and 6 months data for the samples are given in Table 2 below.

TABLE 2

| | | | | Assay | | |
|---|---|---|---|---|---|---|
| | | | | (% content of | Assay | |
| | | description | | ciprofloxacin | (% content of | Total |
| conditions | Months | (*) | pH | HCl) | dexamethasone) | RS** |
| Standard | | To comply | 3.5-6.0 | 90.0-110.0% | 90.0-110.0% | NMT |
| | | | | | | 2.0% |
| Initial | — | complies | 4.21 | 102.05 | 97.86 | 0.240 |
| 25°^{°} C. | 1 | complies | 4.20 | 100.80 | 97.70 | 0.270 |
| | 3 | complies | 4.19 | 104.15 | 98.85 | 0.422 |
| | 6 | complies | 3.98 | 99.72 | 99.34 | 0.420 |
| 40°^{°} C. | 1 | complies | 4.24 | 100.00 | 98.00 | 0.240 |
| | 3 | complies | 4.20 | 106.02 | 98.94 | 0.378 |
| | 6 | complies | 3.62 | 102.28 | 99.05 | 0.500 |
| 50°^{°} C. | 1 | complies | 4.26 | 100.90 | 97.70 | 0.460 |
| | 3 | complies | 4.21 | 107.79 | 96.80 | 0.572 |

(*) complies means complies with the specification and the specification is - clear solutions without any presipitation.
**total RS is total related substances.

1. A clear stable ophthalmic solution comprising (a) an anti-infective agent; (b) a steroidal anti-inflammatory agent; (c) a complexing agent capable of forming an inclusion complex and (d) other pharmaceutically acceptable excipients in a liquid vehicle such that the composition is free of complexation enhancing polymer and such composition when stored at room temperature for one year does not show any precipitation over the storage period.

2. A composition as claimed in claim 1 wherein the anti-infective agent is a quinolone derivative or an aminoglycoside derivative.

3. A composition as claimed in claim 2 wherein the quinolone derivative is ciprofloxacin or its pharmaceutically acceptable salts.

4. A composition as claimed in claim 1 wherein the steroidal anti-inflammatory agent is a corticosteroid.

5. A composition as claimed in claim 4 wherein the corticosteroid is dexamethasone.

6. A composition as claimed in claim 1 wherein the anti-infective agent is present in an amount ranging from about 0.1% w/v to about 30% w/v and the steroidal anti-inflammatory agent is present in an amount ranging from about 0.05% w/v to about 15% w/v.

7. A composition as claimed in claim 3 wherein ciprofloxacin hydrochloride is present in an amount ranging from about 0.1% w/v to about 1.5% w/v.

8. A composition as claimed in claim 5 wherein dexamethasone is present in an amount ranging from about 0.01% w/v to about 10% w/v.

9. A composition as claimed in claim 1 wherein the complexing agent is a β-cyclodextrin ether or polyether selected from dimethyl-β-cyclodextrin, hydroxypropyl-β-cyclodextrin, hydroxyethyl-β-cyclodextrin and mixtures thereof.

10. A composition as claimed in claim 9 wherein the β-cyclodextrin used is hydroxypropyl-β-cyclodextrin.

11. A composition as claimed in claim 10 wherein the hydroxypropyl-β-cyclodextrin is used in an amount ranging from about 0.05% w/v to about 15.0% w/v.

12. A composition as claimed in claim 11 wherein the hydroxypropyl-β-cyclodextrin is used in an amount ranging from about 1.0% w/v to about 10.0% w/v.

13. A composition as claimed in claim 12 wherein the hydroxypropyl-β-cyclodextrin is used in an amount ranging from about 1.5% w/v to about 5.5% w/v.

14. A composition as claimed in claim 1 further comprising a preservative in an amount ranging from about 0.002% w/v to about 0.5% w/v.

15. A composition as claimed in claim 14 wherein the preservative is benzalkonium chloride.

16. A composition as claimed in claim 1 wherein further mannitol is used as an osmogent

17. A composition as claimed in claim 1 further comprising a chelating agent.

18. A composition as claimed in claim 17 wherein the chelating agent is selected from a group comprising edetic acid, edetic acid salts like disodium edetate, sodium edetate, edetate calcium disodium and trisodium edetate, malic acid and mixtures thereof.

19. A composition as claimed in claim 1 wherein the ophthalmic solution has a pH less than 6.5.

20. (canceled)
